(19) United States
(12) Patent Application Publication
(10) Pub. No.: US 2005/0084462 A1
(43) Pub. Date: Apr. 21, 2005

(54) Method for manufacturing a colour mixture for use in food products, pharmaceuticals and cosmetics and colour mixture obtained according to this method

(76) Inventor: Andreas Klingenberg, Reinbek (DE)

Correspondence Address: Friedrich Kueffner, 317 Madison Avenue, Suite 910, New York, NY, 10017, US

(21) Appl. No.: 10/688,773

(22) Filed: Oct. 16, 2003

(51) Int. Cl. A61K 7/021; A23L 1/27; A23B 4/00

(52) U.S. Cl. 424/63; 426/262; 366/349

(57) ABSTRACT

A colour mixture of higher quality, easy to process and usable in food products, pharmaceuticals and cosmetics made of a colour dispersion and a surface active substance, whereby the dispersion contains the colour in a solid which is for example a carotenoid such as carotene and carotenoids of nature identical as well as natural origin, a betanin, a riboflavin, an anthocyanin, a carmine product, a curcuminoid, a porphyerene and/or a chlorophyll compound, a chlorophyllin compound, a copper/chlorophyll and/or copper/chlorophyllin compound.

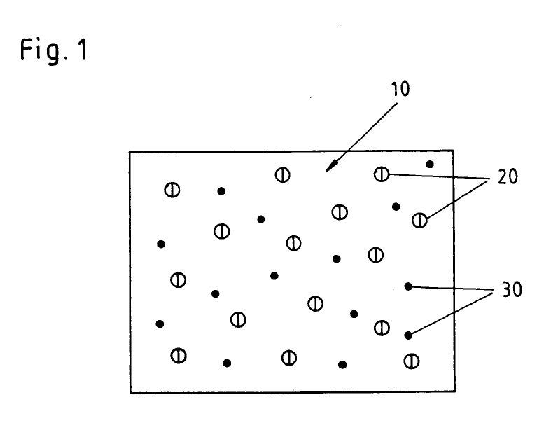

This invention relates to a method for manufacturing a colour mixture for use in food products, pharmaceuticals and cosmetics and a colour mixture obatined according to this method.

Mixtures of colours, in particular in liquid form, are used in many fields for the colour realization of products. Liquid colour mixtures are usual for food products, pharmaceuticals and cosmetics too. This being, special legal provisions are however to be observed such as national provisions or regulations of the European Union. In particular for food products, pharmaceuticals and cosmetics, there are many regulations; the use of colours or colour mixtures requires the certification through the corresponding authorities before being marketed.

For the different operational fields of such mixtures, depending on the product requirements and with respect to the properties of the end product, specific properties of the mixtures are to be considered when they are combined with other components. Properties such as release rate, migration behaviour or light stability, in particular for food products, determine decisively the product quality.

A strong pressure of competition and thus of costs prevails in particular in the field of public health and in the food sector. Moreover, nowadays the consumer requires more and more often products which are attractive and which comply with a high quality standard.

Thus, the aim of this invention is to make available a method for manufacturing a colour mixture which can be easily manufactured, which is of superior quality and which is easy to process, leads to advantageous product properties and for which manufacturing no organic food is used. In particular, an aim of this invention is to make available colour mixtures for food with advantageous properties with respect to release rate, yield, migration behaviour, dissolution rate and light stability.

This aim is achieved by the characteristics indicated in claim 1.

Accordingly, the method according to the invention consists in the fact that in a first stage a colour, a carrier substance and a solvent as well as eventually further constituents are mixed together to a colour dispersion at a temperature of 20° C. to 70° C., that in a second stage the colour dispersion obtained in the first stage is comminuted by means of industrial dispersing and comminuting instruments such as mills, Turrax homogenizers or stirring instruments to a dispersion with a solid with a mean particle size of less than 30 μm in a liquid system, whereby the solid is a carotenoid such as carotene and carotenoids as wells as nature identical as well as of natural origin, a betanin, a riboflavin, an anthocyanine, a carmine product, a curcuminoid, a porphyrene and/or a chlorophyll compound, a chlorophyllin compound, a copper/chlorophyll and/or copper/chlorophyllin compound, that in a third stage a surface active substance is produced at a temperature of 40° C. to 80° C. by an aqueous resolution and that in a fourth stage the surface active substance produced in the third stage of the colour dispersion tempered to 30° C. to 60° C. is added at a temperature of 30° C. to 60° C.

Accordingly, the invention consists in the manufacturing of a superior quality colour mixture usable in food products, pharmaceuticals and cosmetics and easy to be processed which is made of a colour dispersion and a surface active substance, whereby the dispersion contains the colour in a solid which is for example a carotenoid such as carotene and carotenoids of nature identical as well as natural origin, a betanin, a riboflavin, an anthocyanin, a carmine product, a curcuminoid, a porphyerene and/or a chlorophyll compound, a chlorophyllin compound, a copper/chlorophyll and/or copper/chlorophyllin compound.

The properties of the colour mixture obtained according to the method of the invention could be surprisingly greatly optimized and considerably improved by a selection and the combination of an appropriate colour dispersion and an advantageous surface active substance with respect to release rate, yield, migration behaviour, dissolution rate and light stability.

In particular in the colour mixture, the yield in particular could be improved in the respective medium of the end application such as coloured liquid food, for example drink preparations, compared to known colour mixtures. An improved yield is important because the yield is a decisive quality parameter and an improved release behaviour allows for a lower using of product and thus more economical recipes. This results not least in a considerable reduction of costs.

Surprisingly enough, with the colour mixture, an undesirable “marking off” and the transfer of colour between the different phases, for example fat and sugar, in food products could be specifically reduced or even completely avoided. The advantage of the colour mixture is that it is possible with it to distribute a determined colour or a colour combination substantially exclusively in a desired phase and to avoid that it comes to a recognizable phase transfer of the one or the other colour.

Furthermore, the colour mixture shows a quick dissolution in the medium of the end application. This has the advantage that the times for manufacturing can be reduced and thus the course of manufacture optimized and other further processing steps can be adjusted more quickly and efficiently. This results finally also in a saving of machine uptimes and staff expenditure so that a reduction of costs is possible.

Finally, the light stability is a critical point of the known colour mixtures. In particular for natural colour mixtures, the light stability is extremely limited and often does not comply with the requirements for an use, in particular in food products like drinks. This state of facts makes the use of further stabilization measures necessary, through which the for example unsaturated structures are protected against oxidation. Here, the colour mixtures produced according to the method show excellent improvements and allow a reduction of such stabilizers or make the use thereof completely superfluous. On the other side, colours can now be used which could not be used in the past.

Advantageous configurations of the invention are characterized in the subclaims.

It has proved to be particularly advantageous if the colour mixture has a mean particle size of less than 25 μm, in particular preferably of less than 20 μm and most preferably of less than 20 μm.

The colour does exist appropriately in the mixture as solid. The colour can be a colour on natural basis, a modified colour on natural basis or a synthetic colour. Colours on natural basis are most preferred such as carotenoid, betanin, riboflavin, anthocyanin, carmine products, curcuminoid or chlorophyll compounds, chlorophyllin compounds such as copper/chlorophyll and/or copper/chlorophillin compounds, whereby carotenes and carotenoids of nature identical as well as of natural origin fall under carotenoids.

Furthermore, the colour mixture can contain further auxiliary substances and/or additives which are usual in the food industry, the pharmaceutical industry or the cosmetical industry such as enhancers, aromatics or preservatives as well as carrier substances such as polysaccharides, malto dextrines and polyhydric alcohols, and cannot influence negatively the properties of the colour mixture but result in an improvement of the properties.

Preferably, the surface active substance in the colour mixture is contained in a percentage of less than 20%, preferably of less than 10% and most preferably of 1 to 5% referred to the mixture, the percent indications being weight percentages.

Besides said components, further appropriate constituents can be contained in the colour mixture which contribute to a further improvement or to an advantageous appearance of the end product.

The method for manufacturing the colour mixture is carried out as follows.

In a first step a first component, namely a colour dispersion, and in a second step a second component, namely a surface active substance are manufactured. These two components are then homogeneously mixed preferably by being intensively stirred. The mixing takes place preferably at an increased temperature, in particular at 30° to 60° C.

Due to the processing conditions of the method according to the invention, advantageously a mixture is obtained which results in advantageous properties with respect to release rate, migration behaviour or light stability when being used. Add to this that no organic solvent is used for carrying out the method. No oil or fat is used either. Preferably it is started from organic or natural pigments.

In particular the sequence of the individual steps of the method and the selected temperature ranges and the duration of the individual steps of the method have proved to be advantageous for the manufacturing of the colour mixture and for its properties.

A mixing temperature of 30° to 50° C. is particularly preferred for the mixing of the colour dispersion and of the surface active substance, preferably of 40° to 50° C. and a mixing time of 15 to 90 minutes, particularly preferably of 30 to 60 minutes.

It has also been proved advantageous that the colour dispersion has been tempered to 30° to 50° C., by adding the second component, preferably to 35° to 45° C. and most preferably to approximately 40° C.

This being, it is possible to use the colour mixture in all food products, pharmaceuticals or cosmetics. The advantageous properties are achieved in particular in food with a liquid and even also with a solid consistency.

The invention will be illustrated by the following examples.

### EXAMPLE 1

### Manufacturing of the Colour Mixture

1.1 Manufacturing of the Colour Dispersion (Component A)

Approximately 300 g gum arabic will be solved by stirring in a solution of approximately 400 g demineralized water and approximately 100 g malto dextrine at 40° to 50° C. for 30 to 60 minutes until a homogeneous mixture is obtained.

Approximately 100 g curcumin powder are then added and stirred to this mixture. This mixture is ground in a dispersion mill until the mean particle size has reached approximately 10 μm in this suspension.

1.2 Manufacturing of the Surface Active Substance and of the End Product (Component B)

The component A is tempered at approximately 40° C. by stirring and a solution of approximately 100 g water and approximately 10 g Citrem, citric acid ester of monoglycerides (E-472c) are added as emulsifying agent at a temperature of 60° to 80° C. The mixture is stirred further for approximately 30 minutes at approximately 40° to 50° C. The suspension obtained constitutes the colour mixture.

### EXAMPLE 2

The component A manufactured according to example 1 and the colour mixture made of component A and B have now been further examined with respect to their properties.

2.1 The Product Manufactured According to the Invention has an Advantageous Release Behaviour and a Good Yield.

The following method has been developed for calculating the release in order to evaluate and to quantify the release behaviour and the yield of products.

20 to 50 g of the curcumin product to be examined are distributed homogeneously in 100 ml of a 50% suger solution and the colour intensity is determined as L a b value. From the numerical values of the concentration, of the specific absorption value E1% (colour value), of the L and b value, the release is calculated according to the following equation:

$$Release = \frac{b value \cdot 10 [ g \text{/} ml ] \times volume [ ml ]}{L value \times weight [ g ] \times colour value}$$

The examination of both products component A and mixture according to the invention made of component A and B gave the following result:

| | |
|---|---|
| Release of component A: | 20 |
| Release of mixture according to the invention: | 30 |

Thus, the colour mixture shows a release improved by 50% compared with the product (component A). The combination according to the invention of the colour dispersion with a surface active substance is thus superior to the known colour dispersions with respect to their release behaviour.

2.2 The Product Manufactured According to the Invention has an Advantageous Migration Behaviour

The products manufactured according to example 1 have been examined in the following test model with respect to the migration behaviour.

White chocolate coloured with curcumin (0.1 g of the colour mixture or of component A solely according to example 1 to 5 g white chocolate have been compared) has been dropped on moistened filter paper as described under 2.2. Here also it came to a differently strong entrainment of the colour into the filter paper by the mobile phase water. Component A caused a distinct to intensive colouration of the filter paper, whereas the mixture according to the invention coloured only slightly the filter paper.

The colour mixture is thus characterized by clearly improved migration properties.

2.3 The Colour Mixture has an Advantageous Dissolution Rate.

The dissolution rate of component A and of the mixture according to the invention according to example 1 have been examined in an aqueous solution.

To this, 0.1 g curcumin product according to example 1 have been added in 100 ml demineralized water by stiring at 300 rpm with a 5 cm stirring rod at 20° C. in a beaker glass (250 ml). The time to the complete dissolution (no deposit recognizable) of component A or of the colour mixture has been measured. Component A needed 280 to 290 seconds. However, the colour mixture needed only 230 to 240 seconds.

Thus, the colour mixture showed a dissolution behaviour improved by approximately 20% compared to component A.

2.4 The Product Manufactured According to the Method of the Invention has an Advantageous Light Intensity

For determining the light intensity, component A and the colour mixture according to example 1 have been subject to an irradiation in an aqueous solution in a concentration of 20 to 40 mg/250 ml with a “Suntest” device. The colour degradation has been documented by means of a colour tone measurement in the above described L a b system during the whole irradiation duration. The time period until there is a 30% loss of the light intensity (value b) of the aqueous solution has been set as the determining measured variable.

The following measured values have been ascertained for the examined samples:

| | |
|---|---|
| Component A: | 30% colour degradation |
| | after 150 minutes |
| Colour Mixture: | 30% colour degradation |
| | after 290 minutes |

Thus the colour mixture is represented in an enlarged representation, whereby the mixture consisting in a colour dispersion 20 and a surface active substance 30 is indicated by 10.

Accordingly, the invention comprises a colour mixture which can be easily manufactured, which is of superior quality which can be used in food products, pharmaceuticals and cosmetics and which is easy to be processed which is made of a colour dispersion and a surface active substance, whereby the dispersion contains the colour in a solid which is for example a carotenoid such as carotene and carotenoids of nature identical as well as natural origin, a betanin, a riboflavin, an anthocyanin, a carmine product, a curcuminoid, a porphyrene and/or a chlorophyll compound, a chlorophyllin compound, a copper/chlorophyll and/or copper/chlorophyllin compound.

1. A method for manufacturing a colour mixture for use in food products, pharmaceuticals and cosmetics with a high light stability, wherein
in a first stage a colour, a carrier substance and a solvent as well as eventually further constituents are mixed together to a colour dispersion at a temperature of 20° C. to 70° C.,
that in a second stage the colour dispersion obtained in the first stage is comminuted by means of industrial dispersing and comminuting instruments such as mills, Turrax homogenizers or stirring instruments to a dispersion with a solid with a mean particle size of less than 30 μm in a liquid system, whereby the solid is a carotenoid such as carotene and carotenoids as wells as nature identical as well as of natural origin, a betanin, a riboflavin, an anthocyanine, a carmine product, a curcuminoid, a porphyrene and/or a chlorophyll compound, a chlorophyllin compound, a copper/chlorophyll and/or copper/chlorophyllin compound,
that in a third stage a surface active substance is produced at a temperature of 40° C. to 80° C. by an aqueous resolution and
that in a fourth stage the surface active substance produced in the third stage of the colour dispersion tempered to 30° C. to 60° C. is added at a temperature of 30° C. to 60° C.

2. Method according to claim 1, wherein for manufacturing the colour dispersion approximately 300 g gum arabic are solved by stirring in a solution of approximately 400 g demineralized water and approximately 100 g malto dextrine at 40° to 50° C. for 30 to 60 minutes until a homogeneous mixture is obtained, that approximately 100 g curcumin powder are then added and stirred to this mixture, that this mixture is ground in a dispersion mill until the mean particle size has reached approximately 10 μm in this suspension and that, for manufacturing the surface active substance and the end product, the colour dispersion is tempered at approximately 40° C. by stirring and a solution produced at a temperature of 60° to 80° of approximately 100 g water and approximately 10 g Citrem, citric acid ester of monoglycerides (E-472c) is added as emulsifying agent and the mixture is then stirred further for approximately 30 minutes at approximately 40° to 50° C., whereby the suspension obtained constitutes the colour mixture.

3. Method according to claim 1, wherein the auxiliary substance or additive is a sugar, a polysaccharide, a hydrocolloid and/or water.

4. Method according to claim 1, wherein antioxidant agents and/or preservatives are used as auxiliary substances or additives.

5. Method according to claim 1, wherein an emulsifier allowed in food products or food additives such as colours is used as surface active substance.

6. Method according to claim 1, wherein a lecitine, Polysorbate 80, Lactem and/or Citrem is used as surface active substance.

7. Colour mixture manufactured according to the method according to claim 1, wherein it comprises a colour dispersion and a surface active substance.

8. Colour mixture according to claim 7, wherein the dispersion comprises a solid with a mean particle size of less than 30 μm in a liquid system.

9. Colour mixture according to claim 7, wherein the solid is a carotenoid such as carotene and carotenoids of nature identical as well as natural origin, a betanin, a riboflavin, an anthocyanin, a carmine product, a curcuminoid, a porphyerene and/or a chlorophyll compound, a chlorophyllin compound, a copper/chlorophyll and/or copper/chlorophyllin compound.

10. Colour mixture according to claim 7, wherein the dispersion comprises further auxiliary substances and/or additives.

11. Colour mixture according to claim 7, wherein the auxiliary substance or additive is a sugar, a polysaccharide, a hydrocolloid and/or water.

12. Colour mixture according to claim 7, wherein the auxiliary substances or additives are antioxidant agents and/or preservatives.

13. Colour mixture according to claim 7, wherein the surface active substance is an emulsifier or a carrier substance allowed in food products or food additives such as colours.

14. Colour mixture according to claim 7, wherein the surface active substance is a lecithine, Polysorbate 80, Lactem and/or Citrem.
